(19) United States
(12) Patent Application Publication
(10) Pub. No.: US 2011/0052553 A1
(43) Pub. Date: Mar. 3, 2011

(54) STRAIN OF LACTOBACILLUS PLANTARUM LP28 AND ITS USE IN TREATING HYPERSENSITIVITY REACTIONS

(76) Inventors: Tien-Ken HSU, Chiayi County (TW); Cheng-Chih Tsai, Taichung City (TW); Ming-tsai Chu, Kaohsiung County (TW); Po-Chiang Ke, Taichung City (TW); I-Ju Chang, Chiayi City (TW)

(21) Appl. No.: 12/843,428

(22) Filed: Jul. 26, 2010

(30) Foreign Application Priority Data
Aug. 31, 2009 (TW) 098129313

(51) Int. Cl. A61K 35/74; A61P 37/08; C12N 1/20

(52) U.S. Cl. 424/93.45; 435/252.9

(57) ABSTRACT

A new strain of Lactobacillus plantarum LP28, which has deposited at Bioresource Collection and Research Center of Taiwan with a deposited number of BCRC No. 910435, also at China General Microbiological Culture Collection Center (CGMCC), has a deposit number of CGMCC No. 3346. The Lactobacillus plantarum LP28 or its metabolic products are beneficial for allergy releasing, and therefore the Lactobacillus plantarum LP28 is well developed to be used in food products and medications for allergy therapy or hypersensitivity releasing.

### BACKGROUND OF THE INVENTION

1. Field of the Invention

The present invention relates to a Lactobacillus group bacterium, particularly to a Lactobacillus group bacterium with allergy moderating ability.

2. Description of the Related Art

Hypersensitivity (also called hypersensitivity reactions) refers to undesirable reactions produced by the immune system toward some harmless extraneous substances. In recent decades, people who suffer from allergic disorders such as allergic rhinitis, bronchial asthma and atopic dermatitis are dramatically increased. Currently, the therapies of allergic disorders are mainly depending on symptom releasing by specific medications. Nevertheless, most medications for treating allergic disorders may lead to serious side effects. Therefore, it is a crucial issue to develop a new therapy for allergic disorders.

In human body, T-lymphocytes play a central role in cell-mediated immunity. According to their functions and cytokines produced, T-lymphocytes are divided into two major subtypes known as type I lymphocytes (also called Th1 cells) and type II lymphocytes (also called Th2 cells). The main cytokine produced by type I lymphocytes is interferon-γ(IFN-γ). In addition, the main cytokines produced by Th2 cells include interleukin-4 (IL-4) and interleukin-5 (IL-5). Normally, Th1 cells function harmonically with Th2 cells. However, if the reactions of Th1 cells are weaker than that of Th2 cells, it may result in hypersensitivity reaction in human body.

Based on the investigations of some researchers, the harmony of immune system in human body is directly influenced by the ratio between probiotics and pathogens in human gastrointestinal system. Probiotics in human gastrointestinal system play important roles in the protection of gastrointestinal mucosa, the stimulation of immunoglobin synthesis, the secretion of natural antibody and the permeability of intestinal epithelium. On the other hand, the overgrowth of pathogens in human intestine causes damage of the mucosa, and decreases the production of lymphocytes and immunoglobulins, finally inducing the hypersensitivity reactions in human body. It is believed that the microflora in human gastrointestinal tract will deeply influence the development of immune system and its normal functions. Therefore, it may be sufficient to modulate the harmony of intestinal microflora and hypersensitivity reactions by administrating probiotics or prebiotics.

According to studies, lactic acid bacteria (also called LAB) being the major composition of human gastrointestinal microflora carry out plenty reactions to inhibit the propagation of pathogens, also regulate the balance of immune response, scavenge peroxides and lower the serum cholesterol level. Moreover, LAB could produce pleasant flavors and are widely applied to the manufacture of fermented foods. However, in the conventional art, out of the poor resistance to acid environment, most LAB strains are incapable to persist in human digestive system. In this situation, most of the LAB may be ingested and killed by the digestive solution, such as gastric acid and bile salt, and finally be completely excreted out with feces to play any function on immunity.

Consequently, tolerance to gastric acid and bile salt and capability of stimulating Th1 cytokines will be the most important issues for LAB to be a candidate of probiotics for allergy alleviation.

Hence, there is an urgent need of developing a highly resistant Lactobacillus strain to bile salt and gastric acid, for the sake to promote the function of type I lymphocytes in human immune system.

### SUMMARY OF THE INVENTION

The primary objective of this invention is to provide a new strain of Lactobacillus which can promote the secretion of Th1 cytokine interferon-γ in vitro model.

The secondary objective of this invention is to provide a new strain of Lactobacillus which can strongly resistant to gastric acid and bile salt.

Another objective of this invention is to provide a new strain of Lactobacillus which can reduce the hypersensitivity reaction in human body.

An isolated strain of Lactobacillus plantarum LP28 which has deposited at Bioresource Collection and Research Center of Taiwan with a deposited number of BCRC No. 910435, also at China General Microbiological Culture Collection Center (CGMCC) with a deposited number of CGMCC No. 3346.

A therapeutic agent for allergy release comprises an isolated strain of Lactobacillus plantarum LP28 as defined above and the metabolic products of the isolated strain of Lactobacillus plantarum LP28.

A medication for allergy release comprises an isolated strain of Lactobacillus plantarum LP28 as defined above and the metabolic products of the isolated strain of Lactobacillus plantarum LP28.

A food for allergy release comprises an isolated strain of Lactobacillus plantarum LP28 as defined above and the metabolic products of the isolated strain of Lactobacillus plantarum LP28.

Further scope of the applicability of the present invention will become apparent from the detailed description given hereinafter. However, it should be understood that the detailed description and specific examples, while indicating preferable embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a bar chart illustrating the survival rate of Lactobacillus LP28, LF22, LF44, LF46 and LH45 in pH 2 artificial gastric acid;

FIG. 2 is another bar chart illustrating the survival rate of Lactobacillus LP28, LF22, LF44, LF46 and LH45 in pH 3 artificial gastric acid;

FIG. 3 is a bar chart illustrating the growth of Lactobacillus LP28, LF22, LF44, LF46 and LH45 in MRS broth with or without 0.3% bile salt;

FIG. 4 is a bar chart illustrating the effect of Lactobacillus on IFN-γ production by human peripheral blood mononuclear (PBMC) cells;

FIG. 5 is a bar chart illustrating the effect of Lactobacillus on IL-4 production by human peripheral blood mononuclear cells;

FIG. 6 is a bar chart illustrating the ratio of IFN-γ and IL-4.

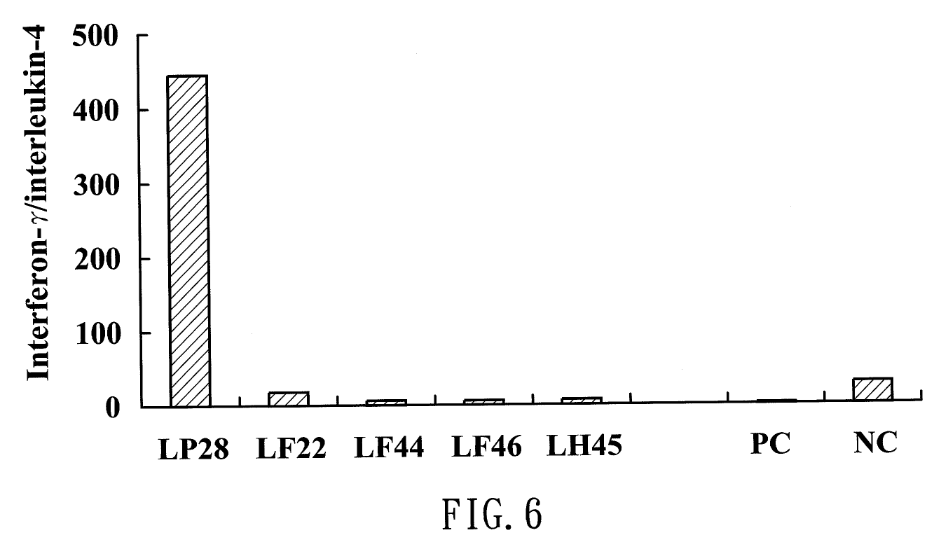

In the various figures of the drawings, the same numerals designate the same or similar parts.

### DETAILED DESCRIPTION OF THE INVENTION

In the present invention, a new strain of Lactobacillus plantarum LP28 with hypersensitivity reducing effect is selected and deposited at Bioresource Collection and Research Center of Taiwan with a deposited number of BCRC No. 910435, also at China General Microbiological Culture Collection Center (CGMCC) with a deposited number of CGMCC No. 3346. It has been proved that the Lactobacillus plantarum LP28 deposited at Bioresource Collection and Research Center of Taiwan (BCRC No. 910435) positively share the same genetic characteristics of the LP28 deposited at China General Microbiological Culture Collection Center (CGMCC No. 3346). The procedure to obtain the Lactobacillus plantarum LP28 is described as follow:

The Screening of the Lactobacillus

In the screening of the Lactobacillus step, some primary lactobacillus isolates were obtained. Bean curds collected from a traditional market were enrichment-cultured at 37° C. for 16-20 hours and some lactobacillus isolates with milk-coagulating ability were selected. Moreover, the lactobacillus isolates showed gram positive and catalase negative were further selected. In the present invention, five lactobacillus strains including one strain of Lactobacillus plantarum, named LP28, three strains of Lactobacillus fermentum, named LF22, LF44 and LF46 respectively, and one strain of Lactobacillus helveticus, named LH45, were obtained.

### The Resistance of Artificial Gastric Acid

The lactobacillus strains (including LP28, LF22, LF44, LF46 and LH45) obtained in the screening of the Lactobacillus step were tested for resistance to acid and bile salt. At first, the lactobacillus strains were subculture in MRS broth at 37° C. for couples of hours, then centrifuged at 5,000 rpm for 5 minutes to get pellets of the lactobacillus strains. After washed and resuspended the pellets of the lactobacillus strains with sterile PBS buffer, aliquots of the bacterial solutions of LP28, LF22, LF44, LF46 and LH45 were added to artificial gastric acid (pH2.0 or pH3.0) and incubated at 37° C. for 2 hours. Cell counts of the LP28, LF22, LF44, LF46 and LH45 were assayed hourly.

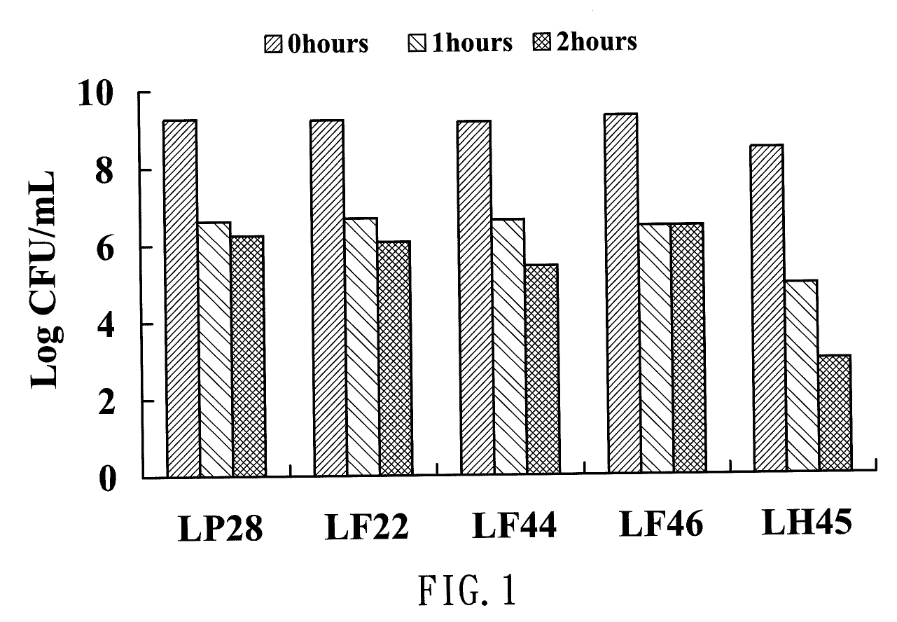

Referring to FIG. 1, LP28, LF22 and LF46 have significant higher survival cell counts than that of LF44 or LH45, especially after 2 hours of incubation in pH 3 artificial gastric acid.

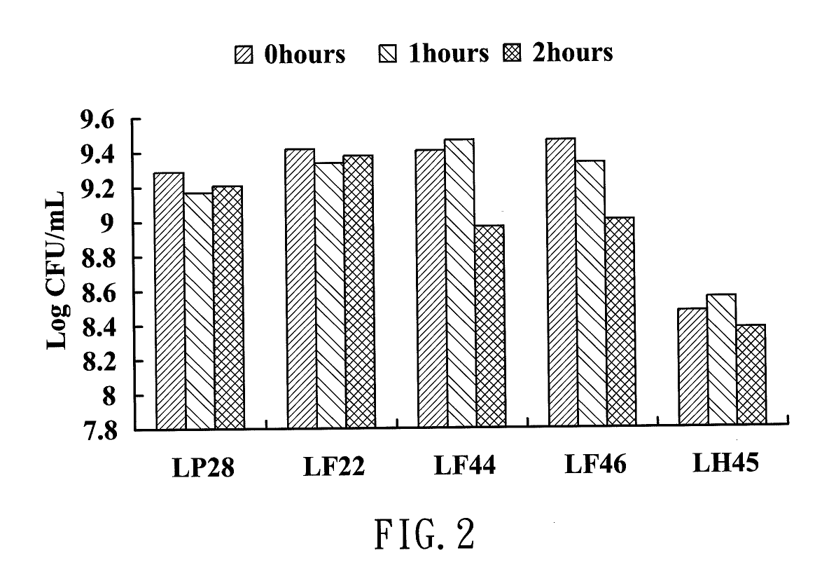

Referring to FIG. 2, LP28 and LF22 showed higher survival rate after 2 hours of incubation in pH 2 artificial gastric acid after 2 hours of incubation.

As a result, it is suggested that the lactobacillus strain of LP28 and LF22 are highly resistant to acid environment than others.

Next, the bacterial solutions of LP28, LF22, LF44, LF46 and LH45 were inoculated in MRS broth with or without 3% bile salt at 37° C. for 16 hours. Cell counts were assayed after incubation.

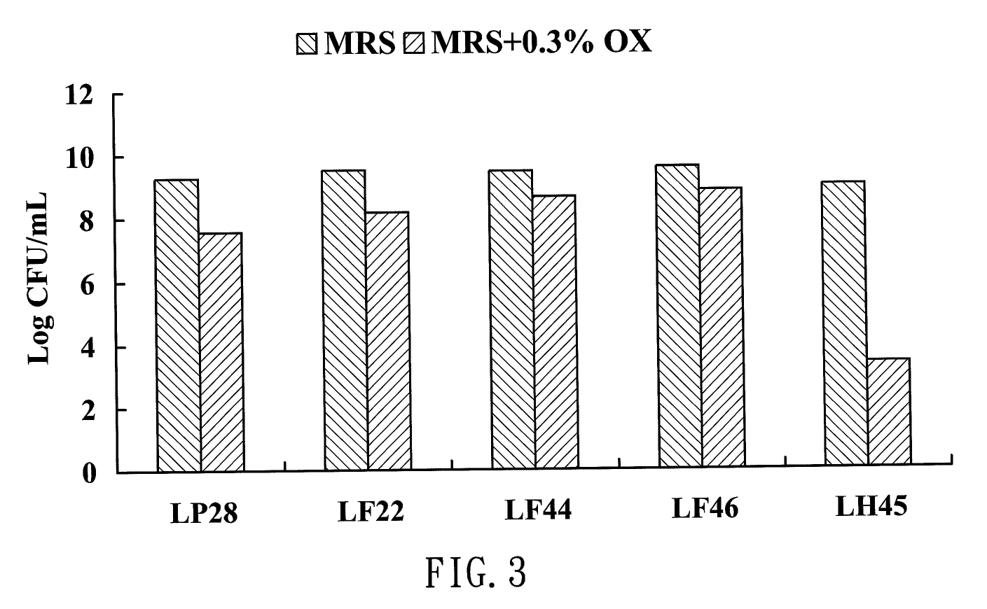

Referring to FIG. 3, the lactobacillus strains but LH45 show great tolerance to bile salt.

### Secretion of Cytokines by Lactobacilli Stimulated Human PBMC

Enzyme-linked immunosorbent assay (ELISA) was used to analyse the Th1 cytokine, IFN-γ, and Th2 cytokine, IL-4, produced by PMBC stimulated by the lactobacillus strains (including LP28, LF22, LF44, LF46 and LH45). First of all, human PBMCs were prepared using density gradient centrifugation from fresh blood of healthy donors. The human PBMCs were washed with Hanks balanced salt solution (HBSS) and resuspended in RPMI-1640 culture medium at a dosage of 2×10⁶ cells per milliliter. Then the lactobacillus strains were added into cultures. In contrast, phosphate buffer saline (PBS) is added as unstimulated control. Cells cultured with 10 ug/ml of phytohemagglutinin (PHA) served as negative control. After incubation, the levels of IFN-γ and IL-4 were quantified in cell free supernatants by means of ELISA.

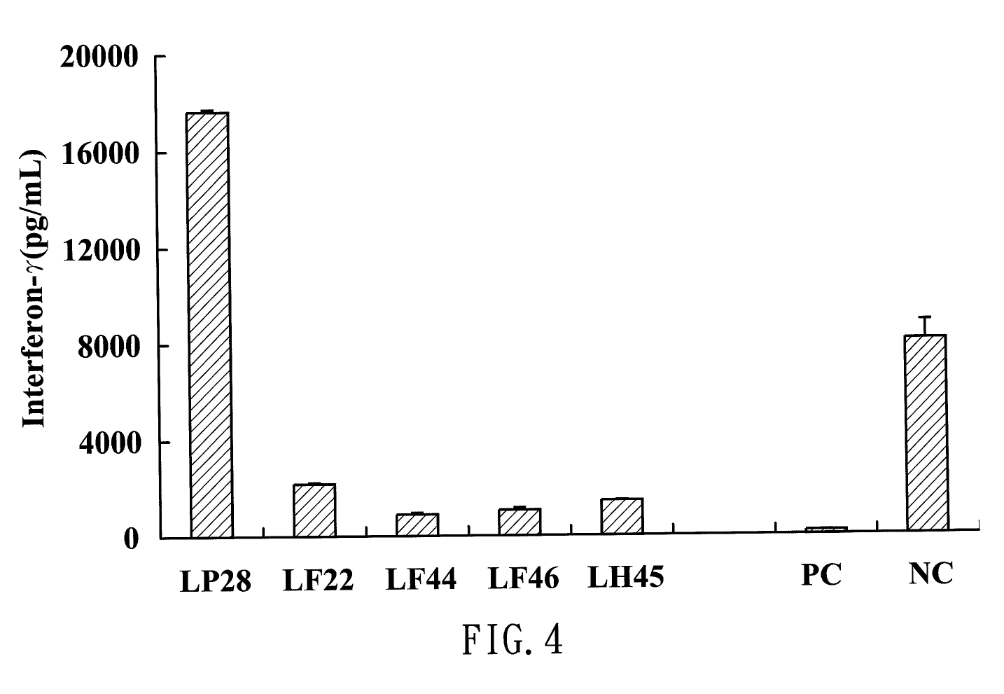

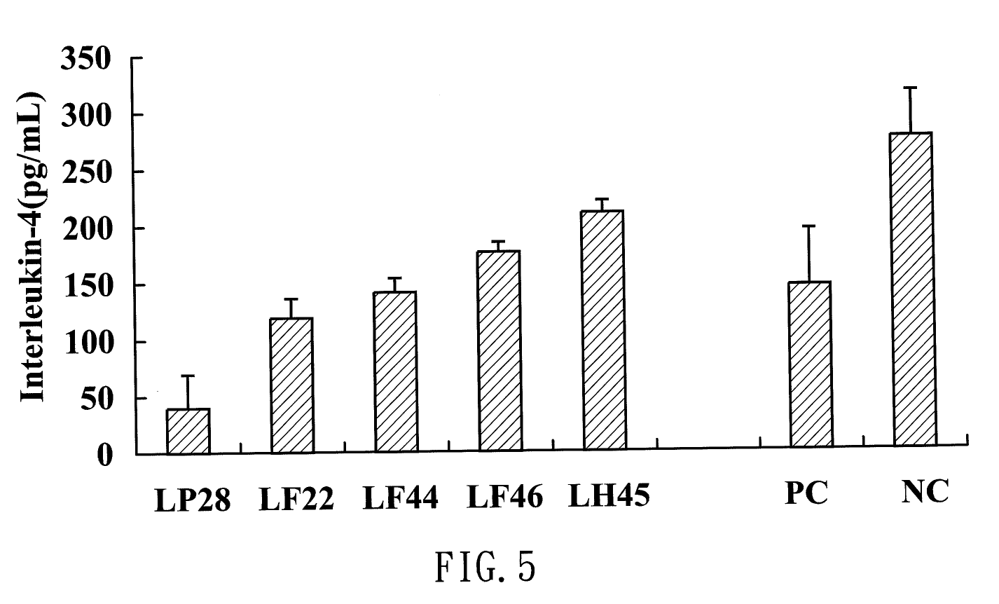

Referring to FIG. 4 and FIG. 5, LP28 shows superior great ability to stimulate the production of INF-γ (FIG. 4) and reduce the production of IL-4 (FIG. 5) of PBMCs. INF-γ will stimulate the formation of Th1 lymphocytes, further harmonizing the balance between the Th1 and Th2 response, also reducing hypersensitivity reaction in human. IL-4 is the cytokine responsible for the formation of IgE-producing cells. The binding of IgE to mast cells will trigger off a serial signal transductions to induce the release of chemicals (like histamine), and causes inflammatory reactions and allergic symptoms. In summarization of the data showed above, the LP28 performed well in leveling off the concentration of IL-4 and balance the Th1 and Th2 response of cytokines (including interferon-γ and interleukin-4) production.

According to the data described above, it is obvious that the LP28 positively stimulates the production of Th1 cytokine, IFN-γ, and negatively controls the production of Th2 cytokine, IL-4; therefore reduces the hypersensitivity reaction in human. Moreover, it is believed that the metabolic products of LP28 could be used as additives into food products and medications, anti-allergy yoghurt for example, for moderating the hypersensitivity reaction in human.

Although the invention has been described in detail with reference to its presently preferred embodiment, it will be understood by one of ordinary skill in the art that various modifications can be made without departing from the spirit and the scope of the invention, as set forth in the appended claims.

What is claimed is:

1. An isolated strain of Lactobacillus plantarum LP28 which has deposited at Bioresource Collection and Research Center of Taiwan with a deposited number of BCRC No. 910435, also at China General Microbiological Culture Collection Center (CGMCC), with a deposited number of CGMCC No. 3346.

2. The isolated strain of Lactobacillus plantarum LP28 as defined in claim 1, which can stimulates the secretion of Th1 cytokine, balancing the interaction between the type I lymphocytes and type II lymphocytes.

3. The isolated strain of Lactobacillus plantarum LP28 as defined in claim 2, wherein the cytokine is Interferon-γ.

4. The isolated strain of Lactobacillus plantarum LP28 as defined in claim 1, which can reduce the secretion of Th2 cytokine, balancing the interaction between the type I lymphocytes and type II lymphocytes.

5. The isolated strain of Lactobacillus plantarum LP28 as defined in claim 4, wherein the cytokine is Interleukin-4.

6. A therapeutic agent for allergy releasing, comprising:
an isolated strain of Lactobacillus plantarum LP28 as defined in claim 1; and
metabolic products of the isolated strain of Lactobacillus plantarum LP28 as defined in claim 1.

7. A medication for allergy releasing, comprising:
an isolated strain of Lactobacillus plantarum LP28 as defined in claim 1; and
metabolic products of the isolated strain of Lactobacillus plantarum LP28 as defined in claim 1.

8. A food for allergy releasing, comprising:
an isolated strain of Lactobacillus plantarum LP28 as defined in claim 1; and
metabolic products of the isolated strain of Lactobacillus plantarum LP28 as defined in claim 1.

9. The food for allergy releasing as defined in claim 8, wherein the food is fermented milk.
